(19) United States
(12) Patent Application Publication
(10) Pub. No.: US 2025/0020653 A1
(43) Pub. Date: Jan. 16, 2025

(54) Method for Detecting Cancer in a Patient

(71) Applicant: Nordic Bioscience A/S, Herlev (DK)

(72) Inventors: Nicolas Willumsen, Dyssegard (DK); Anne-Christine Anne-Christine, København S (DK); Morten Karsdal, København Ø (DK)

(73) Assignee: Nordic Bioscience A/S, Herlev (DK)

(21) Appl. No.: 18/708,017

(22) PCT Filed: Nov. 8, 2022
(86) PCT No.: PCT/EP2022/081178

§ 371 Date: May 7, 2024

(30) Foreign Application Priority Data
Nov. 8, 2021 (GB) 2116020.5

(51) Int. Cl. G01N 33/574; G01N 33/566; G01N 33/58; G01N 33/68

(52) U.S. Cl. CPC G01N 33/57488; G01N 33/566; G01N 33/58; G01N 33/6854; G01N 33/6887; G01N 2333/78

(57) ABSTRACT

Described herein are methods of immunoassay for detecting and/or monitoring cancer in a patient. A sample from the patient is contacted with denatured type III collagen. The amount of binding between the denatured type III collagen and antibodies in the sample that specifically recognise and bind to denatured type III collagen is then detected and determined, and the amount of binding is correlated with values associated with normal healthy subjects, and/or with values associated with known disease severity, and/or with values obtained from the patient at a previous time point, and/or with a predetermined cut-off value.

### FIELD OF INVENTION

The present invention relates to a method of immunoassay for detecting and/or monitoring cancer in a patient.

### BACKGROUND

Autoimmune diseases, such as rheumatoid arthritis (RA), and cancer are two major health problems worldwide with challenging clinical circumstances for patients. Both diseases are driven by an imbalance and dysregulation of the immune system. However, whilst RA is associated with excessive immune activation, cancer patients typically have a supressed immune response. In autoimmune diseases, autoreactive lymphocytes and the initiation of the production of autoantibodies against self-epitopes and auto-antigens leads to an abnormal and unwanted immune response (in an otherwise functional tissue or organ) with associated tissue destruction and, ultimately, loss of organ function (1)(2). In cancer, the immune system is suppressed and lymphocytes that should normally eliminate the tumor present with an exhausted and inactive phenotype (3)(4). This may allow a primary tumor and cancer cells to grow uncontrolled, metastasize to other organs, and, in many cases, death is the final consequence.

One of the major overlapping and essential processes of autoimmune diseases and cancer is connective tissue remodelling resulting from fibrosis, a state of chronic inflammation, and ongoing wound healing processes (5)(6). The enhanced and chronic connective tissue turnover manifests by increased synthesis and degradation of extracellular matrix proteins, of which collagens are major components (5)(6). This phenomenon, that characterizes most patients with autoimmune diseases, may lead to a vicious cycle with the development of autoreactivity and autoantibodies against collagen products, thereby maintaining unwanted inflammation and tissue destruction (5). It has been shown that patients with RA have increased levels of circulating antibodies against type I and II collagen (7)(8)(9) but also against citrullinated forms of type I and II collagen (10)(11)(12). Despite increased connective tissue turnover in the tumor microenvironment, few studies have investigated how immune cells such as B cells respond to the accumulation of collagens in cancer tissues however.

Svobodova et al (13) described the use of an indirect ELISA assay for the detection of antibodies to native human type I, II and III in serum of patients with Juvenile Chronic Arthritis (JCA) and RA. The study reported significantly higher antibody titres to all three collagen types in comparison with age-matched healthy individuals. Moreover, average titres in patients with RA were higher than in JCA patients.

Trentham et al (14) described that mononuclear cells from patients with Rheumatoid Arthritis exhibit cellular sensitivity in vitro in response to human type II and III collagens leading to cell proliferation, but no cellular response was reported to denatured alpha chains of these collagens.

Elkayam et al (15) described the T cell immune responses of 51 patients with Rheumatoid Arthritis and healthy controls to human collagen II and III. It was found that 57% of patients with RA and 27% of healthy controls proliferated to collagen III response. A lower percentage of RA patients—38% and 17% of healthy controls had proliferative responses to collagen 1l.

Fehr et al. (16) suggested that native and denatured collagen type 1, II and III are autoantigens capable of inducing T cell activation in patients with RA, that the synovial tissue of RA patients to some degree synthesizes antibodies directed against native and denatured collagens 1, II and III, and that all these antibodies seem to participate in the immune complex formation in RA joints and to activate the complement system.

Kresina et al (17) described experiments investigating cell-mediated immune responses to native and denatured type I, II and III collagen using an IgG-induced animal model of immune synovitis. In vitro exposure of splenic cell cultures from synovitic animals to native collagen 1, 11 and III antigens showed cellular reactivity of T cell origin with an incidence of: Type I, 43%; Type II, 43%; Type III, 57%. A higher incidence of immune responses was observed to denatured collagens in cultures of the same animals: Type I, 50%; Type II, 50%; Type III, 67%.

Tishler et al (18) described the detection of antibodies to native human type I and Ill collagen by radioimmunoassay in serum samples from 42 patients with Rheumatoid Arthritis. The authors reported that 38% of investigated patients had significantly elevated antibody titres. Moreover, there was a significant association between the presence of collagen antibodies and HLA-DR4.

Stuart et al (19) described the presence of antibodies to native and denatured type I, 1l, Ill, IV and V collagens in serum and synovial fluid from patients with Rheumatoid Arthritis using radioimmunoassay. Mean levels of binding to all collagens by sere from 30 RA patients were significantly higher than those from 20 normal subjects. Denaturation of collagens resulted in increased binding by RA serum. Binding to denatured type III collagen was over 2-fold higher comparing to native collagen III.

Menzel et al (20) described a strong cross-reactivity of anti-type I antibodies found in synovial fluid from RA patients with denatured type III collagen. The study used radioimmunoassay as a detection method.

Beard et al (21) described the minor or negative presence of antibodies against native and denatured collagen 1, 11 and III in patients with rheumatoid arthritis and chronic low back pain using the heamagglutination method. The authors reported weak reactions against denatured collagen I and II in 30-40% of the tested sera. Reactions to native and denatured collagen Ill were largely negative in sera from all patients

A study by Ingrosso et al. (22) has shown that autoantibodies against type I and Ill collagen are elevated in prostate cancer patients compared to healthy controls.

A study by Fernandez-Madrid et al (23) has shown that serum levels of autoantibodies against type 1, 11, 111, and V collagen are significantly higher in lung cancer patients compared to healthy controls.

### SUMMARY

The present inventors have developed a novel immunoassay for quantifying the amount of anti-denatured type III collagen antibodies (Anti-dCol3) in a sample, and have shown that the levels of Anti-dCol3 are lower in samples from patients with various types of cancer than in healthy controls. The inventors have also shown that the levels of anti-denatured type II collagen antibodies (Anti-dCol2) in samples from patients with ovarian or stomach cancer are lower than in healthy controls.

Accordingly, in a first aspect, the present invention provides a method of immunoassay for detecting and/or monitoring cancer in a patient, the method comprising:
i) contacting a sample from a patient with denatured type III collagen;
ii) detecting and determining the amount of binding between said denatured type III collagen and antibodies in the sample that specifically recognise and bind to denatured type III collagen (such antibodies being referred to herein as “anti-denatured type III collagen antibodies” or “Anti-dCol3”); and
iii) correlating the amount of binding between said denatured type III collagen and said antibodies as determined in step (ii) with values associated with normal healthy subjects, and/or with values associated with known disease severity, and/or with values obtained from said patient at a previous time point, and/or with a predetermined cut-off value.

Preferably, the immunoassay is a solid phase immunoassay in which in step (i) the denatured type III collagen is bound to a solid support. Preferably in step (ii) the amount of binding between the denatured type III collagen and antibodies in the sample that specifically recognise and bind to denatured type III collagen is detected and determined by:
a) adding a detection antibody, wherein said detection antibody is specifically reactive with antibodies from the sample; and
b) detecting and determining the amount of binding between the detection antibody and antibodies from the sample bound to the denatured type III collagen on the solid support.

In preferred embodiments, the detection antibody is specifically reactive with human immunoglobin G (IgG) antibodies. For example, the detection antibody may specifically recognise and bind to a human IgG constant region. The detection antibody may for example be a monoclonal antibody. The detection antibody may for example be radiolabelled, be linked to a fluorophore, or be an enzyme linked antibody.

In preferred embodiments, the cancer is bladder cancer, breast cancer, colorectal cancer, head and neck cancer, kidney cancer, liver cancer, lung cancer, melanoma, ovarian cancer, pancreatic cancer, prostate cancer, or stomach cancer.

The sample from a patient is preferably a biofluid. The biofluid may be, but is not limited to, serum, plasma, urine or a supernatant from cell or tissue cultures. Preferably the biofluid is serum or plasma. Preferably the patient is a human (and hence the patient sample is human patient sample).

The immunoassay may be but is not limited to a radioimmunoassay, fluorescence immunoassay, or an enzyme-linked immunosorbent assay.

In a second aspect, the present invention provides a method of immunoassay for detecting and/or monitoring ovarian or stomach cancer in a patient, the method comprising:
i) contacting a sample from a patient with denatured type II collagen; ii) detecting and determining the amount of binding between said denatured type II collagen and antibodies in the sample specifically recognise and bind to denatured type II collagen (such antibodies being referred to herein as “anti-denatured type II collagen antibodies” or “Anti-dCol2”); and
ii) correlating said amount of binding between said denatured type II collagen and said antibodies as determined in step (ii) with values associated with normal healthy subjects, and/or with values associated with known disease severity, and/or with values obtained from said patient at a previous time point, and/or with a predetermined cut-off value.

Preferably, the immunoassay is a solid phase immunoassay in which in step (i) the denatured type II collagen is bound to a solid support. Preferably in step (ii) the amount of binding between the denatured type II collagen and antibodies in the sample that specifically recognise and bind to denatured type II collagen is detected and determined by:
a) adding detection antibody, wherein said detection antibody is specifically reactive with antibodies from the sample; and
b) detecting and determining the amount of binding between the detection antibody and antibodies from the sample bound to the denatured type II collagen on the solid support.

In preferred embodiments, the detection antibody is specifically reactive with human immunoglobin G (IgG) antibodies. For example, the detection antibody may specifically recognise and bind to a human IgG constant region. The detection antibody may for example be a monoclonal antibody. The detection antibody may for example be radiolabelled, be linked to a fluorophore, or be an enzyme linked antibody. The sample from a patient is preferably a biofluid. The biofluid may be, but is not limited to, serum, plasma, urine or a supernatant from cell or tissue cultures. Preferably the biofluid is serum or plasma. Preferably the patient is a human (and hence the patient sample is human patient sample).

The immunoassay may be but is not limited to a radioimmunoassay, fluorescence immunoassay, or an enzyme-linked immunosorbent assay.

As used herein the term “amount of binding” refers, in relation to the amount of binding between denatured type II or III collagen and antibodies in a sample, to the quantification of binding between said denatured collagen and said antibodies in the patient sample. Likewise, in relation to the amount of binding between a detection antibody and antibodies from a sample that are bound to denatured type II or III collagen bound to a solid support, it refers to the quantification of binding between said detection antibody and said antibodies from the patient sample (which in turn therefore correlates to the amount of binding between the denatured collagen and the antibodies in the patient sample). In the Examples set out below, an ELISA method is used in which spectrophotometric analysis is used to measure the amount of binding. However, any suitable analytical method can be used.

As used herein the term “predetermined cut-off value” means an amount of binding that is determined statistically to be indicative of a high likelihood of a disease (i.e. a cancer) or a particular severity thereof in a patient, in that a measured value of the target peptide in a patient sample that is at or above the statistical cut-off value corresponds to at least a 70% probability, preferably at least an 75% probability, more preferably at least an 80% probability, more preferably at least an 85% probability, more preferably at least a 90% probability, and most preferably at least a 95% probability of the presence of said disease or said particular severity thereof.

As used herein, the term “values associated with normal healthy subjects” means standardised quantities of binding determined by the method described supra for samples from subjects considered to be healthy, i.e. without disease (or, more specifically, without cancer); and the term “values associated with known disease severity or prognosis” means standardised quantities of binding determined by the method described supra for samples from patients known to have disease (i.e. a cancer) of a known severity.

As used herein the term “monoclonal antibody” refers to both whole antibodies and to fragments thereof that retain the binding specificity of the whole antibody, such as for example a Fab fragment, F(ab′)2 fragment, single chain Fv fragment, or other such fragments known to those skilled in the art. As is well known, whole antibodies typically have a “Y-shaped” structure of two identical pairs of polypeptide chains, each pair made up of one “light” and one “heavy” chain. The N-terminal regions of each light chain and heavy chain contain the variable region, while the C-terminal portions of each of the heavy and light chains make up the constant region. The variable region comprises three complementarity determining regions (CDRs), which are primarily responsible for antigen recognition. The constant region allows the antibody to recruit cells and molecules of the immune system. Antibody fragments retaining binding specificity comprise at least the CDRs and sufficient parts of the rest of the variable region to retain said binding specificity.

In the methods of the present invention, a monoclonal antibody comprising any constant region known in the art can be used. In the case of mouse antibodies and human antibodies, the constant light chains are classified as either kappa or lambda light chains. Heavy constant chains are classified as mu, delta, gamma, alpha, or epsilon, and define the antibody's isotype as IgM, IgD, IgG, IgA, and IgE, respectively. The IgG isotype has several subclasses, including, but not limited to IgG1, IgG2, IgG3, and IgG4 in the case of humans and IgG1, IgG2a, IgG2b, IgG2c and IgG3 in the case of mice. The monoclonal antibody may preferably be of the IgG isotype, including any one of the IgG subclasses.

The CDR of an antibody can be determined using methods known in the art such as that described by Kabat et al. Antibodies can be generated from B cell clones. The isotype of the antibody can be determined by ELISA specific for IgM, IgG or IgA isotype, or subclass. The amino acid sequence of the antibodies generated can be determined using standard techniques. For example, RNA can be isolated from the cells, and used to generate cDNA by reverse transcription. The cDNA is then subjected to PCR using primers which amplify the heavy and light chains of the antibody. For example primers specific for the leader sequence for all VH (variable heavy chain) sequences can be used together with primers that bind to a sequence located in the constant region of the isotype which has been previously determined. The light chain can be amplified using primers which bind to the 3′ end of the Kappa or Lamda chain together with primers which anneal to the V kappa or V lambda leader sequence. The full length heavy and light chains can be generated and sequenced.

In a third aspect, the present invention provides a method of treating cancer in a patient in need thereof, the method comprising:
(a) carrying out a method of immunoassay in accordance with the first or second aspect of the present invention on a sample from a patient in order to detect whether the patient has cancer; and
(b) administering to the patient a therapy for the treatment of the cancer if it is determined in step (a) that the patient has cancer.

The therapy may be any therapy suitable for treating the cancer in question. The therapy may for example comprise or consist of one or more surgeries, one or more radiation therapies, one or more medicaments (such as for example one or more chemotherapies, one or more immunotherapies and/or one or more hormonal therapies), or combinations thereof. Medicaments may be formulated to topical or systemic administration. Topical medicaments may for example be formulated as creams, foams, gels, lotions, or ointments for administration. Systemic medicaments may for example be formulated for enteral or parenteral administration. Surgeries may be curative surgeries, preventative surgeries, debulking surgeries, palliative surgeries and/or restorative surgeries.

For example, where the cancer is bladder cancer, suitable therapies may comprise one or more of: transurethral resection of bladder tumor (TURBT) with or without intravesical chemotherapy or immunotherapy; radical cystectomy plus neoadjuvant chemotherapy or transurethral resection with chemoradiation or partial cystectomy plus neoadjuvant chemotherapy; cisplatin-based chemotherapy, optionally followed by radical cystectomy or chemoradiation; carboplatin-based chemotherapy; immune checkpoint inhibitors; radical cystectomy; and palliative radiotherapy.

Where the cancer is breast cancer, suitable therapies may for example comprise one or more of: a mastectomy, quadrantectomy or lumpectomy; estrogen receptor blockers (such as tamoxifen); aromatase inhibitors (such as anastrozole or letrozole) that block production of estrogen; CDK inhibitors; one or more chemotherapeutic agents such as a combination of cyclophosphamide, doxorubicin and optionally a taxane (such as docetaxel), or a combination of cyclophosphamide, methotrexate, and fluorouracil; one or more monoclonal antibodies such as trastuzumab and/or pertuzumab; and radiotherapy.

Where the cancer is colorectal cancer, suitable therapies may for example comprise one or more of: endoscopic mucosal resection or endoscopic submucosal dissection; a partial colectomy (or proctocolectomy for rectal lesions); chemotherapy agents such as for example capecitabine, fluorouracil, irinotecan, oxaliplatin or UFT; antiangiogenic drugs such as for example bevacizumab; epidermal growth factor receptor inhibitors, such as for example aflibercept, cetuximab and panitumumab; radiation therapy; immune checkpoint inhibitors; and monoclonal antibodies such as pembrolizumab or dostarlimab.

Where the cancer is head and neck cancer, suitable therapies may for example comprise one or more of: surgery, including but not limited to laser surgery; radiation therapy, including but not limited to 3D conformal radiation therapy, intensity-modulated radiation therapy, particle beam therapy and brachytherapy; one or more chemotherapy agents such as for example, paclitaxel, carboplatin, cetuximab, docetaxel, cisplatin and fluorouracil; photodynamic therapy utilizing amphinex; monoclonal antibodies such as cetuximab, bevacizumab, erlotinib, pembrolizumab or nivolumab; gene therapies such as gendicine; and immune checkpoint inhibitors.

Where the cancer is kidney cancer, suitable therapies may for example comprise one or more of: whole kidney removal or partial removal of the kidney, by surgery; freezing the tumour or treating it with high temperatures; biological therapies such as everolimus, torisel, nexavar, sutent, axitinib, sunitinib, pazopanib, sorafenib, cabozantinib and/or lenvatinib; immunotherapy using interferon and/or interleukin-2; monoclonal antibodies such as nivolumab; immune checkpoint inhibitors; and radiotherapy.

Where the cancer is liver cancer, suitable therapies may for example comprise one or more of: partial surgical resection; liver transplantation; percutaneous ablation, comprising injecting chemicals into the liver (ethanol or acetic acid) or producing extremes of temperature using radio frequency ablation, microwaves, lasers or cryotherapy; transarterial chemoembolization; targeted therapy using sorafenib; transarterial radioembolization; internal radiotherapy; photodynamic therapy; chemotherapies such as gemcitabine and/or cisplatin; and palliative radiotherapy.

Where the cancer is lung cancer, suitable therapies may for example comprise one or more of: surgery, such as performing a lobectomy, a sublobar excision (wedge resection) or removal of a whole lung (pneumonectomy); radiotherapy, examples of which include radiotherapy given together with chemotherapy, post-operative radiotherapy, brachytherapy (localized radiotherapy) given directly inside the airway, prophylactic cranial irradiation, stereotactic radiation and palliative radiotherapy; chemotherapy using for example one more agents such as cisplatin/carboplatin, etoposide, gemcitabine, paclitaxel, docetaxel, vinorelbine, topotecan, irinotecan and pemetrexed; epidermal growth factor receptor (EGFR) inhibitors drugs such as erlotinib, gefitinib, afatinib, dacomitinib or osimertinib; and immunotherapy, using for example anti PD-L1 monoclonal antibodies such as atezolizumab, nivolumab or pembrolizumab, monoclonal antibodies targeting cytotoxic T-lymphocyte-associated protein 4 (CTLA-4) such as ipilimumab, and/or monoclonal antibodies that targets vascular endothelial growth factor such as bevacizumab.

Where the cancer is melanoma, suitable therapies may for example comprise one or more of: surgical excision of the tumour, and optionally lymph nodes in the area of the tumour; interferon treatment; chemotherapy using agents such as for example dacarbazine; small-molecule targeted therapies using for example BRAF inhibitors (such as vemurafenib and dabrafenib), MEK inhibitors (trametinib), C-Kit inhibitors and/or NRAS inhibitors; immunotherapy using cytokines (e.g. IL-2 and/or IFN-α), immune check point inhibitors (such as for example anti-CTLA-4 monoclonal antibodies such as ipilimumab or tremelimumab, toll-like receptor (TLR) agonists, CD40 agonists, anti-PD-1 antibodies such as pembrolizumab, pidilizumab or nivolumab, LAG-3 inhibitors such as relatlimab, and/or PD-L1 antibodies), and/or adoptive cell transfer (using for example pre-stimulated, modified T cells or dendritic cells); and radiotherapy.

Where the cancer is ovarian cancer, suitable therapies may for example comprise one or more of: removal of one (unilateral oophorectomy) or both ovaries (bilateral oophorectomy), and optionally also the fallopian tubes (salpingectomy), uterus (hysterectomy) and/or the omentum (omentectomy); debulking surgery; chemotherapy (including neoadjuvant or adjuvant chemotherapy) using agents such as paclitaxel, cisplatin, topotecan, doxorubicin, epirubicin, gemcitabine, carboplatin, docetaxel, vincristine, dactinomycin, etoposide, cyclophosphamide, oxaliplatin or combinations thereof; radiotherapy; hormonal therapy; and immunotherapy, such as for example the antibody drug bevacizumab.

Where the cancer is pancreatic cancer, suitable therapies may for example comprise one or more of: surgical resection; chemotherapy using agents such as gemcitabine, 5-FU, erlotinib, FOLFIRINOX, nab-paclitaxel or combinations thereof; radiotherapy; the somatostatin analog class of medications; lanreotide; targeted therapy using everolimus or sunitinib; nuclear medicine therapy with radiolabeled peptides or hormones such as iobenguane; and techniques such as radiofrequency ablation (RFA), cryoablation, or hepatic artery embolization.

Where the cancer is prostate cancer, suitable therapies may for example comprise one or more of: radiotherapy; chemotherapy using chemotherapeutic agents, such as for example docetaxel, cabazitaxel, docetaxel, thalidomide, and combinations thereof; immunotherapy, such as for example the monoclonal antibody bevacizumab; hormonal therapies such as for example abiraterone and enzalutamide; external beam radiation therapy; particle therapy; high-intensity focused ultrasound; cryotherapy; and surgical procedures such as for example a radical prostatectomy.

Where the cancer is stomach cancer, suitable therapies may for example comprise one or more of: surgical procedures such as for example an endoscopic mucosal resection. endoscopic submucosal dissection or gastrectomy; chemotherapy using agents such as fluorouracil, capecitabine, BCNU, methyl-CCNU, and doxorubicin, mitomycin C, cisplatin, Taxotere or combinations thereof; targeted therapy using epidermal growth factor receptor 2 inhibitors, such as trastuzumab; and radiotherapy.

### FIGURES

FIG. 1: Assay sensitivity and specificity. (A) The assay is more sensitivity to denatured type III collagen vs native type III collagen. Albumin has been introduced as a non-sense control. The presence of autoantibodies against type III collagen (Anti-dCol3) was investigated in serum samples from patients with Rheumatoid Arthritis (RA1-RA5) at a serum dilution of 1:100. (B) Levels of Anti-dCol3 were higher in RA patients compared to healthy controls. Data are displayed as mean chemiluminescence (RLU/sec).

FIG. 2: Overnight incubation test of serum samples with respective proteins. Samples were spiked with an equal molecular ratio of native/denatured collagen III and albumin. Values on X-axis are given as measured luminescence (RLU/sec) and correspond to the number of bound anti-denatured type III collagen antibodies after overnight incubation with respective proteins at serum dilution 1:100. Data are presented as a mean luminescence signal.

FIG. 3: Anti-dCol3 levels were significantly decreased in 12 types of cancer compared to healthy controls. Anti-dCol3 (A) and anti-dCol2 (B) levels in patients with bladder cancer (n=20), breast cancer (n=20), colorectal cancer (n=20), head and neck cancer (n=20), kidney cancer (n=20), liver cancer (n=3), lung cancer (n=20), melanoma (n=20), ovarian cancer (n=20), pancreatic cancer (n=20), prostate cancer (n=20), and stomach cancer (n=20) were compared to healthy controls (n=33). Biomarker levels are presented as Tukey box plots and statistical differences were analysed using the Kruskal-Wallis test adjusted for Dunn's multiple comparisons test. *:p<0.05, **:p<0.01, ***:p<0.001, ****:p<0.0001.

FIG. 4: ROC analysis of (A) Anti-dCol3 and (B) Anti-dCol2 and identification of diagnostic power for discrimination between cancer patients and healthy controls.

### EXAMPLES

### Materials and Methods

### Characterization of the Monoclonal Antibodies

The developed Anti-dCol3 and Anti-dCol2 assays both utilize a commercial mouse monoclonal anti-human HRP-labelled antibody of the IgG1 isotype (Abcam, #ab99759). The antibody is used for the specific detection of IgG autoantibodies targeting denatured type II or type III collagen in human serum. According to the manufacturer, Abcam antibodies specifically react with the Fc portion of the heavy chain of all subclasses of human IgG (IgG1-IgG4) as demonstrated by ELISA. In addition, the antibodies demonstrated no levels of cross-reactivity with other antibody isotypes. The antibodies were purified using protein G chromatography and displayed a high level of purity.

### Development and Procedure of the Anti-dCol3 and Anti-dCol2 Assays

The development of the Anti-dCol3 and Anti-dCol2 assays was preceded by initial checkerboard experiments aimed to find the most optimal reagents, protein concentrations, reaction time, and temperature. The optimized ELISA protocols for (A) the Anti-dCol3 assay and (B) the Anti-dCol2 assay are comprised of the following steps: (A) Native Human Type III Collagen (Abcam, #ab7535) or (B) ELISA Grade Human Type II Collagen (Chondrex) was initially pre-heated in the Eppendorf ThermoMixer for 15 min at 72° C. Pre-heated collagen was subsequently diluted in carbonate coating buffer (pH=9.5) to a final concentration of 1 μg/ml. A 96-well white microplate (Nunc MaxiSorp™, ThermoFisher) was coated with 100 μl/well of prepared (A) type III collagen or (B) type II collagen for 20 hours at 4° C. with shaking at 300 rounds per minute (rpm). The plate was subsequently washed five times with washing buffer (25 mM Tris, 50 mM NaCl, 0.1% Tween20, pH=7.2) using an automated microplate washer and blocked with 100 μl/well of Chonblock blocking buffer (Chondrex) for 2 hours at 20° C. with 300 rpm shaking. After completed incubation, the plate was washed five times with washing buffer. Serum samples were diluted (1:100) in assay buffer (50 mM PBS-BTB, 8 g NaCl, pH=7.4) and 100 μl of each were applied in duplicates together with standard and controls and incubated for 2 hours at 20° C. with 300 rpm shaking.

The plate was again washed five times with washing buffer. Further, 100 μl/well of mouse monoclonal anti-human IgG HRP-antibodies (Abcam, #ab99759) at a concentration of 200 ng/ml diluted in assay buffer were applied and incubated for 1 hour at 20° C. with 300 rpm shaking. After completed incubation, the plate was washed five times with washing buffer. Subsequently, 100 μl/well of prepared BM Chemiluminescence ELISA POD substrate (Roche) was added and the plate was immediately placed in the Fluoroskan Microplate Reader (Thermofisher).

The plate was read with Thermo Scientific Skanit software version 6.0.1 (Thermofisher) in the luminescence mode and with the following parameters: 1 min of shaking at 240 rpm (medium speed), 2 min of pause, and measurement time 1000 ms. A standard curve was plotted using a 5-parameter logistic curve fit.

### Technical Evaluation of the Anti-dCol3 and Anti-dCol2 Assays

The technical performance of the Anti-dCol3 and Anti-dCol2 assays was performed in compliance with the international guidelines and Good Research Practice (GRP) procedures developed by Nordic Bioscience (Herlev, Denmark), defining standard processes and procedures for assay development and acceptance. The reproducibility of the assays was established by determining the inter-and intra-assay coefficients of variation (CV). According to validation procedures, inter-assay variation was acceptable if CV was 15% and Intra-assay variation was ≤10%. The technical validation of the developed assay was established based on 10 independent day-to-day runs including standard, 2 assay controls (one below and one above assay EC50), and 5 lot-to-lot samples covering the entire analytical measurement range. Quantification of antibodies against denatured type III collagen and denatured type II collagen in human serum relied on the developed 2-fold diluted 7-point standard curve (Anti-dCol3) or 2-fold diluted 6-point standard curve. Both, the standards, and controls have been developed from the native serum material from RA patients with high disease activity. The measurement range of the assays was defined as the lower limit of measurement range (LLMR) and the upper limit of measurement range (ULMR) and calculated from 10 day-to-day independent runs. As no international reference units exist for the quantification of antibodies against collagen in serum, the calibration was performed in Relative Units (RU/mL). Each analytical run was accepted only if all standard points displayed CV 10% and percentage of relative error (% RE) 15% within the analytical measurement range. Similarly, all assay controls needed to display CV 20% with a calculated concentration within the target range of mean±20%. All tested samples were run in double determinations and accepted if CV was 20%. The linearity of the assays was determined by dilution recovery tests and serial dilutions (2-fold) of 4 serum samples. The protein inhibition test was assessed by spiking different concentrations of collagen into serum samples to investigate its impact on the serum matrix. The analyte stability was assessed by temperature treatment and repeatable four freeze and thaw cycles of 3 serum samples. Temperature treatment involved serum incubation at 0, 2, 4, 24, and 48 hours at either 4° C. or 20° C. Moreover, analytical interference with common serum components was assessed by the addition of low/high concentrations of hemoglobin (2.5/5 mg/mL), lipids (1.5/5 mg/mL), or biotin (3.0/9.0 ng/mL) to serum samples. Untreated serum was used as a reference point for the calculation of the recovery percentage.

### Clinical Evaluation of the Autoimmunity Assays

The biological relevance of the Anti-dCol3 and Anti-dCol2 assays was assessed in patients with different solid tumor types (Table 1), in a few RA patients, and compared to healthy controls. The measurements of both biomarkers were performed according to the procedures described above.

The cohort of patients with different solid tumor types consisted of 223 subjects comprising patients with bladder cancer (n=20), breast cancer (n=20), colorectal cancer (n=20), head and neck cancer (n=20), kidney cancer (n=20), liver cancer (n=3), lung cancer (n=20), melanoma (n=20), ovarian cancer (n=20), pancreatic cancer (n=20), prostate cancer (n=20), and stomach cancer (n=20). All cancer samples were obtained from Proteogenex (Los Angeles, CA, USA). Additionally, a control cohort involved age-matched healthy controls (n=33) obtained from BioIVT (Westbury, NY, USA) was included. Serum samples were collected pre-treatment. Cohort demographics are shown in Table 1.

TABLE 1
Cohort demographics of cancer patients and healthy controls

| | Healthy | Cancer | Total |
|---|---|---|---|
| | (N = 33) | (N = 223) | (N = 256) |
| Diagnosis | | | |
| Healthy | 33 (100%) | 0 (0%) | 33 (12.9%) |
| Bladder cancer | 0 (0%) | 20 (9.0%) | 20 (7.8%) |
| Breast cancer | 0 (0%) | 20 (9.0%) | 20 (7.8%) |
| Colorectal cancer | 0 (0%) | 20 (9.0%) | 20 (7.8%) |
| Head and neck cancer | 0 (0%) | 20 (9.0%) | 20 (7.8%) |
| Kidney cancer | 0 (0%) | 20 (9.0%) | 20 (7.8%) |
| Liver cancer | 0 (0%) | 3 (1.3%) | 3 (1.2%) |
| Lung cancer | 0 (0%) | 20 (9.0%) | 20 (7.8%) |
| Melanoma | 0 (0%) | 20 (9.0%) | 20 (7.8%) |
| Ovarian cancer | 0 (0%) | 20 (9.0%) | 20 (7.8%) |
| Pancreatic cancer | 0 (0%) | 20 (9.0%) | 20 (7.8%) |
| Prostate cancer | 0 (0%) | 20 (9.0%) | 20 (7.8%) |
| Stomach cancer | 0 (0%) | 20 (9.0%) | 20 (7.8%) |
| Stages | | | |
| I | 0 (0%) | 7 (3.1%) | 7 (2.7%) |
| II | 0 (0%) | 49 (22.0%) | 49 (19.1%) |
| III | 0 (0%) | 93 (41.7%) | 93 (36.3%) |
| IV | 0 (0%) | 74 (33.2%) | 74 (28.9%) |
| Missing | 33 (100%) | 0 (0%) | 33 (12.9%) |
| Age (years) | | | |
| Mean (SD) | 57.7 (5.69) | 59.3 (11.2) | 59.1 (10.7) |
| Median [Min, Max] | 57.0 | 61.0 | 60.0 |
| | [49.0, 69.0] | [30.0, 87.0] | [30.0, 87.0] |
| Missing | 0 (0%) | 1 (0.4%) | 1 (0.4%) |
| Sex | | | |
| Male | 21 (63.6%) | 121 (54.3%) | 142 (55.5%) |
| Female | 12 (36.4%) | 102 (45.7%) | 114 (44.5%) |
| Ethnicity | | | |
| Black | 13 (39.4%) | 0 (0%) | 13 (5.1%) |
| Caucasian | 11 (33.3%) | 223 (100%) | 234 (91.4%) |
| Hispanic | 9 (27.3%) | 0 (0%) | 9 (3.5%) |

### Statistical Analysis

The difference between healthy controls and the different cancer groups was tested using the nonparametric Kruskal-Wallis test with Dunn's multiple comparisons test. AUROC analysis was performed to evaluate the diagnostic power for discriminating between cancer patients and healthy controls. A P-value of <0.05 was considered statistically significant. All graphical presentations were performed using GraphPad version 8 (GraphPad software, Inc., CA, USA) and statistical analyses with MedCalc statistical software version 19.3 (Medcalc software, Belgium).

### Results

### Technical Evaluation of the Developed Anti-dCol3 and Anti-dCol2 Assays

The technical performance of the Anti-dCol3 and Anti-dCol2 assays was evaluated based on 10 independent day-to-day assay runs and presented in Table 2. According to that, inter-and intra-assay variations were determined to be 15% and 7% respectively for Anti-dCol3 and 13% and 3% respectively for Anti-dCol2 (acceptance criteria <15% for inter-and <10% for intra-assay variation). The detection range was determined to 2.84-96.53 RU/mL for Anti-dCol3 and 3.72-96.72 RU/mL for Anti-dCol2 (corrected for pre-dilution). Both assays displayed dilution recovery linearity within the measurement range of human serum from initial serum dilution to a 1:32 dilution for Anti-dCol3 and to a 1:8 for Anti-dCol2. The average concordance of dilution-factor-corrected results for the serum samples was 103% (94%-119%) for Anti-dCol3 and 88% (79%-124%) for Anti-dCol2.

The analyte stability was acceptable after 4 freeze-thaw cycles with a mean analyte recovery of 98% for Anti-dCol3 and 95% for Anti-dCol2. The analyte recovery for Anti-dCol3 was acceptable for 24 and 48 hours once stored at 4° C. (97% and 88% respectively). The analyte recovery for Anti-dCol2 was acceptable for at 4° C. and at 20° C. (84% and 80% respectively) and 48 hours at 4° C. (92%). The prolonged storage of HRP-detection antibodies was acceptable for at least 24 hours at 20° C. and 37° C. with a mean stress recovery of 107% for both assays. No interference was detected from low or high contents of lipids with a mean recovery of 89% and 81% for Anti-dCol3 and 98% and 110% for Anit-dCol2, for low or high respectively. Furthermore, Anti-dCol3 was checked additionally for interference with biotin, and no interference was observed with low and high biotin concentrations with a mean recovery of 91% and 80% respectively. Importantly, both assays showed increased interference with haemoglobin, exceeding the acceptable mean range, meaning that measured serum must be free from haemoglobin content prior to use.

TABLE 2
Technical performance of the Anti-dCol3

| | RESULTS |
|---|---|
| ASSAY PARAMETERS | Anti-dCol3/Anti-dCol2 |
| Inter-assay variation (mean) | 15%/13% |
| Intra-assay variation (mean) | 7%/3% |
| Measurement range (RU/mL) | 2.84-96.53/3.73-96.72 |
| LLMR-ULMR | |
| EC50 (mean) | 40.78/38.55 |
| Std A concentration (RU/mL) | 100 |
| Slope (mean) | 1.03/1.03 |
| CO1 (RU/mL); (mean ± 20%) | 61.2 (49.0-73.4) / 68.4 (54.7-82.1) |
| CO2 (RU/mL); (mean ± 20%) | 29.7 (23.8-35.6) / 39.1(31.3-46.9) |
| Dilution Recovery of human serum | 103%/88% |
| Analyte Recovery 24 h, | [97%][123%]/[84%][80%] |
| [4° C.][20° C.] | |
| Analyte Recovery 48 h, | [88%][135%]/[92%][74%] |
| [4° C.][20° C.] | |
| Analyte Recovery, 4 freeze-thaw | 98%/95% |
| cycles | |
| Antibody Stress Recovery 24 h, | [95%][107%] |
| [20° C.][37° C.] | |
| Hemoglobin Recovery [Low][High] | >120%/>120% |
| Lipids Recovery [Low][High] | [89%][81%]/[98%][110%] |
| Biotin Recovery [Low][High] | [91%][80%]/NA |

### Assays Sensitivity and Specificity

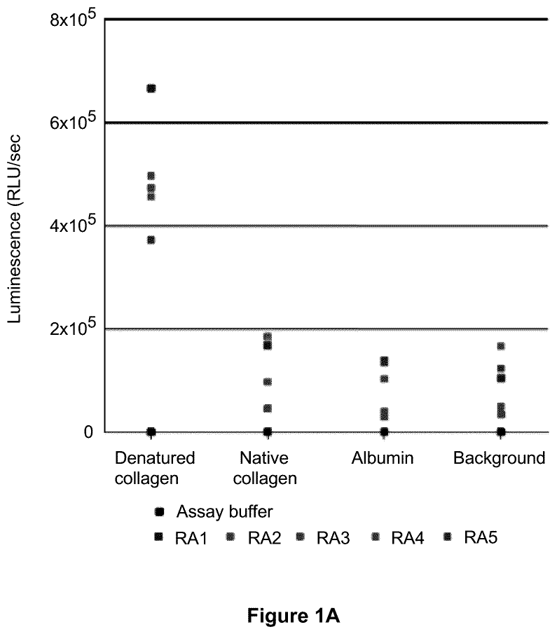

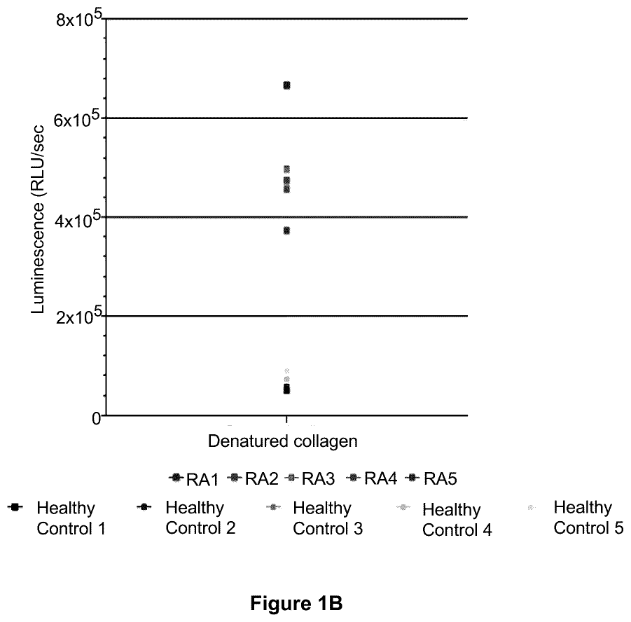

Thermal denaturation of the collagens increased considerably the sensitivity of the assays compared to native proteins. Testing of serum samples from patients with Rheumatoid Arthritis (RA1-RA5) displayed substantially higher binding of autoantibodies to denatured rather than native collagen (FIG. 1A). Denaturation of type III collagen resulted in increased binding of autoantibodies measured with the Anti-dCol3 assay for each tested RA serum sample with >3.5-fold increase for RA1, >10.5-fold for RA2, >2.5-fold for RA3, >4.5-fold for RA4, and >2-fold for RA5 compared with native collagen III (FIG. 1A). The mean increase in sensitivity for tested patients was >4.5-fold. The same increase was observed for autoantibodies against denatured type II collagen measured with the Anti-dCol2 assay. More specifically, denaturation of type II collagen resulted in >2-fold increase for RA1, >3.5-fold for RA2, >2-fold for RA3 and >2-fold for RA4 (data not shown), giving a mean increase in sensitivity for tested patients of almost 2.5-fold. For both assays, a non-sense control including albumin was tested with a molecular ratio equal to collagen, and a marginal binding was observed in RA serum samples for the Anti-dCol3 assay (FIG. 1A) and the Anti-dCol2 assay (data not shown). In addition, both assays displayed a low background noise of serum samples once incubated alone without a protein coater.

The mean levels of autoantibodies against denatured type III collagen were 7.5-fold higher in RA patients compared to healthy controls (FIG. 1B).

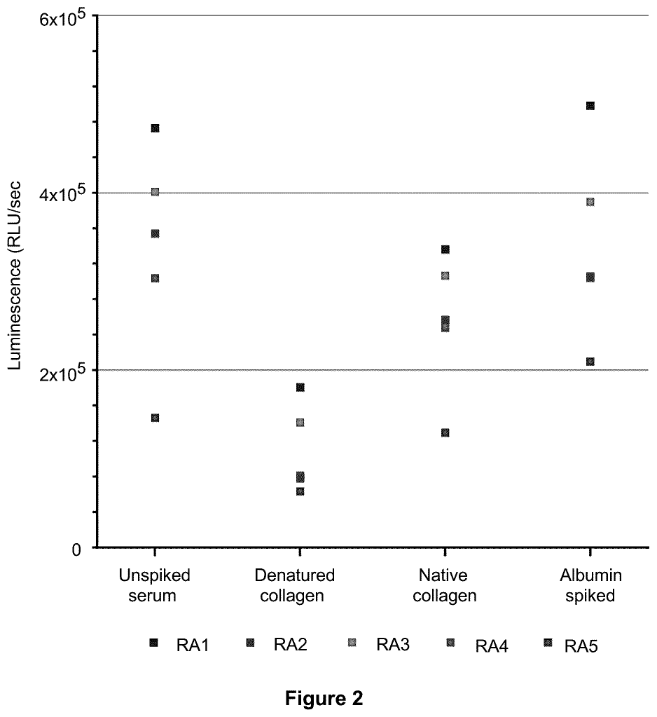

The specificity and increased sensitivity of the developed assays for detection of anti-collagen antibodies was investigated by overnight incubation of 5 serum samples from patients with Rheumatoid Arthritis (RA1-RA5) with equal molecular ratios (2×1013/ml molecules) of denatured collagen, native collagen, and albumin (FIG. 2). The hypothesis behind this experiment was that incubation of RA serum containing anti-collagen antibodies with denatured collagen will lead to the excessive binding of these antibodies to injected collagen, resulting in decreased ELISA chemiluminescence values (signal). Incubation with native collagen was introduced as a control, and albumin was included as a non-sense control.

In the case of Anti-dCol3, there was a great difference in signal decrease dependent on serum spiking with either native or denatured collagen III and compared to unspiked serum. Signal decrease for native and denatured collagen III, was 34% vs 62% for RA1, 31% vs 78% for RA2, 25% vs 65% for RA3, 20% vs 74% for RA4 and 26% vs 57% for RA5, respectively (FIG. 2). The overall mean signal difference for the samples was 27% vs 67% decrease for native and denatured type collagen III respectively, suggesting more extensive anti-collagen III autoantibodies binding to the denatured rather native protein. Interestingly, serum spiking with albumin did not result in a signal decrease for any of the tested samples, suggesting assay specificity for the detection of anti-collagen III autoantibodies.

Similarly, the Anti-dCol2 assay displayed a substantial decrease in measured signal after serum spiking with native or denatured collagen II and compared to unspiked serum. Consequently, a signal decrease for native and denatured collagen II was 22% vs 39% for RA1, 7% vs 9% for RA2, and 15% vs 26% for RA3, respectively (data not shown). The overall mean signal difference for the samples was a 15% vs 33% decrease for native and denatured collagen II respectively, suggesting similar to the Anti-dCol3 assay that there is a more extensive binding of anti-collagen II autoantibodies to denatured rather than native protein. Importantly, also for Anti-dCol2 assay, serum spiking with albumin did not result in a signal decrease for any of the tested samples, suggesting assay specificity for detection of anti-collagen II autoantibodies.

### Biological Evaluation of the Anti-dCol2 and Anti-dCol3 Assay in Cancer

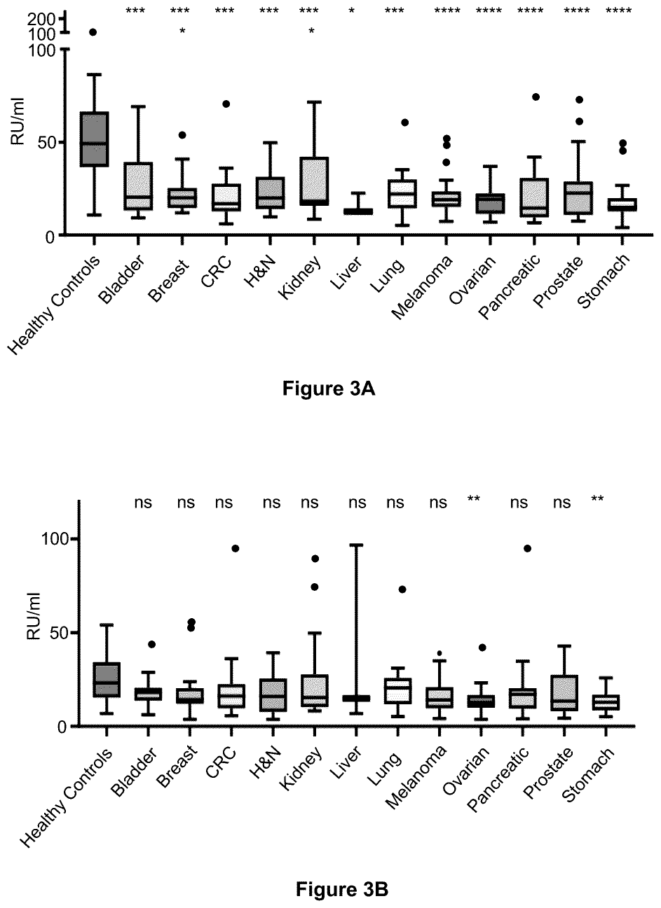

Interestingly, when assessing anti-dCol3, anti-dCol3 levels in serum were significantly decreased in bladder cancer (p=0.0007), breast cancer (p=0.0002), colorectal cancer (p<0.0001), head and neck cancer (p=0.0005), kidney cancer (p=0.005), liver cancer (p=0.030), lung cancer (p=0.0004), melanoma (p<0.0001), ovarian cancer (p<0.0001), pancreatic cancer (p<0.0001), prostate cancer (p<0.0001) and stomach cancer (p<0.0001) compared to healthy controls (FIG. 3A).

Conversely, anti-dCol2 levels were not significantly different in patients with bladder cancer (p>0.9999), breast cancer (p=0.199), colorectal cancer (p=0.250), head and neck cancer (p=0.138), kidney cancer (p>0.9999), liver cancer (p>0.9999), lung cancer (p>0.9999), melanoma (p=0.111), pancreatic cancer (p=0.149) and prostate cancer (p=0.062) compared to healthy controls (FIG. 3B). However, anti-dCol2 was decreased in ovarian cancer (p=0.006) and stomach cancer (p=0.002) compared to healthy controls.

### The Diagnostic Power of Anti-dCol3 for Separating Cancer Patients and Healthy Controls

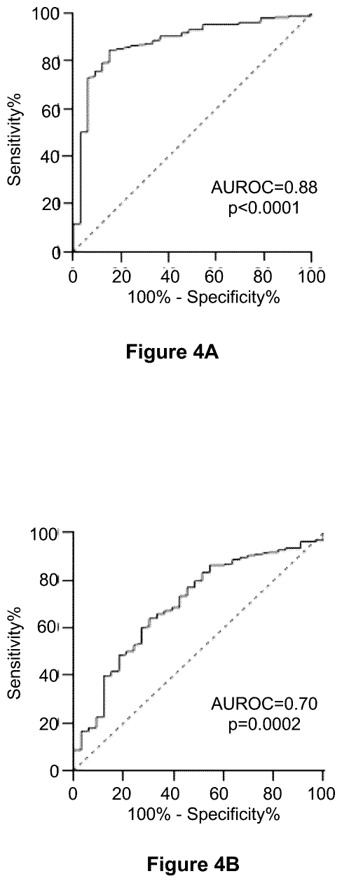

To investigate the ability of Anti-dCol3 for discriminating between cancer patients and controls a ROC analysis was performed. The ROC analysis was done using 223 cancer vs. 33 healthy controls. The Anti-dCol3 assay provided a high diagnostic power for discriminating between cancer and healthy with an AUROC of 88%, p<0.0001 (FIG. 4A). The ROC analysis was also performed on the Anti-dCol2 measurements to compare the discriminative performance of the two assays. Anti-dCol2 had an AUROC of 70%, p=0.0002 (FIG. 4B).

### CONCLUSIONS

The inventors have shown that the Anti-dCol3 and Anti-dCol2 assays only detect denatured collagen (respectively, denatured collagen type III and denatured collagen type II) and not native collagen. Furthermore, that autoimmunity against type III collagen (major connective tissue collagen) is higher in RA patients compared to healthy controls, while anti-dCol3 is significantly lower in 12 different solid tumor types compared to healthy controls suggesting opposing direction in ‘self-recognition’, clean-up of self-antigen, and immune dysregulation between autoimmunity and cancer. Interestingly, while it has been shown that autoimmunity against type II collagen (anti-dCol2) is higher in RA patients [24], in the present study anti-dCol2 was not significantly lower in all solid tumor types compared to healthy controls suggesting that the decreased levels of autoimmunity in cancer are specific for type III collagen.

In this specification, unless expressly otherwise indicated, the word ‘or’ is used in the sense of an operator that returns a true value when either or both of the stated conditions is met, as opposed to the operator ‘exclusive or’ which requires that only one of the conditions is met. The word ‘comprising’ is used to mean ‘including or consisting of’. All prior teachings acknowledged above are hereby incorporated by reference. No acknowledgement of any prior published document herein should be taken to be an admission or representation that the teaching thereof was common general knowledge in Australia or elsewhere at the date hereof.

### REFERENCES

1. Ludwig R J, Vanhoorelbeke K, Leypoldt F, Kaya Z, Bieber K, McLachlan S M, et al. Mechanisms of autoantibody-induced pathology. Vol. 8, Frontiers in Immunology. 2017.
2. Aggarwal A, Srivastava R, Agrawal S. T cell responses to citrullinated self-peptides in patients with rheumatoid arthritis. Rheumatol Int. 2013; 33(9):2359-63.
3. Shimizu K, Iyoda T, Okada M, Yamasaki S, Fujii S I. Immune suppression and reversal of the suppressive tumor microenvironment. Vol. 30, International Immunology. 2018. p. 445-55.
4. Seliger B, Massa C. Immune therapy resistance and immune escape of tumors.

Vol. 13, Cancers. 2021. p. 1-16.
5. Karsdal M A, Kraus V B, Shevell D, Bay-Jensen A C, Schattenberg J, Rambabu Surabattula R, et al. Profiling and targeting connective tissue remodeling in autoimmunity—A novel paradigm for diagnosing and treating chronic diseases. Vol. 20, Autoimmunity Reviews. 2021.
6. Nissen N I, Karsdal M, Willumsen N. Collagens and Cancer associated fibroblasts in the reactive stroma and its relation to Cancer biology. J Exp Clin Cancer Res. 2019;38(1):115.
7. Durlik-Popińska K, Żarnowiec P, Lechowicz L, Gawȩda J, Kaca W. Antibodies isolated from rheumatoid arthritis patients against lysine-containing Proteus mirabilis O3 (S1959) lipopolysaccharide may react with collagen type I. Int J Mol Sci. 2020; 21(24):1-15.
8. Vinci C, Infantino M, Raturi S, Tindell A, Topping L M, Strollo R, et al. Immunoglobulin A antibodies to oxidized collagen type II as a potential biomarker for the stratification of spondyloarthritis from rheumatoid arthritis. Scand J Rheumatol. 2020; 49(4):281-91.
9. Manivel V A, Mullazehi M, Padyukov L, Westerlind H, Klareskog L, Alfredsson L, et al. Anticollagen type II antibodies are associated with an acute onset rheumatoid arthritis phenotype and prognosticate lower degree of inflammation during 5 years follow-up. Ann Rheum Dis. 2017; 76(9):1529-36.
10. Suzuki A, Yamada R, Ohtake-Yamanaka M, Okazaki Y, Sawada T, Yamamoto K. Anti-citrullinated collagen type I antibody is a target of autoimmunity in rheumatoid arthritis. Biochem Biophys Res Commun. 2005; 333(2):418-26.
11. Koivula M K, Âman S, Karjalainen A, Hakala M, Risteli J. Are there autoantibodies reacting against citrullinated peptides derived from type I and type II collagens in patients with rheumatoid arthritis? Ann Rheum Dis. 2005;64(10):1443-50.
12. Yoshida M, Tsuji M, Kurosaka D, Kurosaka D, Yasuda J, Ito Y, et al. Autoimmunity to citrullinated type II collagen in rheumatoid arthritis. Mod Rheumatol. 2006; 16(5):276-81.
13. Svobodova J., Beckvar R., Novotna J. et al. Anticollagen antibodies in patients with Juvenile chronic arthritis. Clinical Rheumatology 1988; vol. 7, no. 3, pp. 378-383.
14. Trentham D E., Dynesius R A., Rocklin R E. et al. Cellular Sensitivity to Collagen in Rheumatoid Arthritis. The New England Journal of Medicine 1978; vol. 299, no. 7, pp. 327-332.
15. Elkayam O., Zinger H., Zisman E. et al. Regulatory T cell activity specific to human II and Ill collagens in Rheumatoid Arthritis. J Rheumatol. 1991; vol. 18, no. 4, pp. 516-512.
16. Fehr K. Autoimmune reactions and rheumatoid arthritis. European Journal of Rheumatology and Inflammation 1982; vol 5, no. 4, pp. 439-456.
17. Kresina T F., Rosner I A., Goldberg V M. et al. Immunoglobulin G-Induced Experimental Chronic Immune Synovitis: Cell-Mediated Immunity to Native Interstitial Collagen Molecules and Their Constituent Polypeptide Chains. Cellular Immunology 1984; vol. 87, pp. 504-516.
18. Tishler M., Brautbar C., Yaron M. et al. Association between HLA-DR4 and production of collagen-specific antibodies in Israeli patients with rheumatoid arthritis.

Isr J Med Sci. 1986; vol. 22, no. 11, pp. 771-773.
19. Stuart J M., Huffstutter E H., Townes A S. et al. Incidence and specificity of antibodies to type I, II, III, IV and V collagen in rheumatoid arthritis and other rheumatoid diseases as measured by 1251-Radioimmunoassay. Arthritis and Rheumatism 1983; vol. 26, no. 7, pp. 832-840.
20. Menzel J., Steffen C., Kolarz G. et al. Demonstration of anticollagen antibodies in rheumatoid arthritis synovial fluids by 14C-radioimmunoassay. Arthritis and Rheumatism 1978; vol. 21, no. 2, pp. 243-248.
21. Beard H K., Lea D J., Ryvar R. Anomalous reactions in the haemagglutination assay for ant-collagen antibodies studies on patients with rheumatoid arthritis or chronic low back pain. Journal of Immunological Methods 1979; vol. 31, pp. 119-128.
22. Ingrosso G, Fantini M, Nardi A, Benvenuto M, Sacchetti P, Masuelli L, et al. Local radiotherapy increases the level of autoantibodies to ribosomal P0 protein but not to heat shock proteins, extracellular matrix molecules and EGFR/ErbB2 receptors in prostate cancer patients. Oncol Rep. 2013; 29(3):1167-74.
23. Fernandez-Madrid F, Karvonen R L, Kraut M J, Czelusniak B, Ager J W. Autoimmunity to collagen in human lung cancer. Cancer Res. 1996; 56(1):121-6.
24. Drobinski P, Aighobahi E, Bay-Jensen A C, Karsdal M. POS0473 Autoimmunity against collagen in rheumatoid arthritis (RA): Serum levels of IgG against denatured type II collagen significantly elevated in RA. Ann Rheum Dis. 2021;80(Suppl 1):468.1-468.

1: A method of immunoassay for detecting and/or monitoring cancer in a patient, the method comprising:
i) contacting a sample from a patient with denatured type III collagen;
ii) detecting and determining the amount of binding between said denatured type III collagen and antibodies in the sample that specifically recognise and bind to denatured type III collagen; and
iii) correlating said amount of binding between said denatured type III collagen and said antibodies as determined in step (ii) with values associated with normal healthy subjects, and/or with values associated with known disease severity, and/or with values obtained from said patient at a previous time point, and/or with a predetermined cut-off value.

2: The method of claim 1, wherein the immunoassay is a solid phase immunoassay wherein in step (i) the denatured type III collagen is bound to a solid support.

3: The method of claim 2, wherein step (ii) comprises detecting and determining the amount of binding between the denatured type III collagen and antibodies in the sample that specifically recognise and bind to denatured type III collagen by:
a) adding a detection antibody, wherein said detection antibody is specifically reactive with antibodies from the sample; and
b) detecting and determining the amount of binding between the detection antibody and antibodies from the sample bound to the denatured type III collagen on the solid support.

4: The method of claim 3, wherein the patient is a human patient and the detection antibody is an antibody specifically reactive with human immunoglobin G (IgG) antibodies.

5: The method of claim 4, wherein the detection antibody is a monoclonal antibody.

6: The method of claim 3, wherein the detection antibody is radiolabelled, is linked to a fluorophore or is an enzyme-linked antibody.

7: The method of claim 1, wherein the cancer is bladder cancer, breast cancer, colorectal cancer, head and neck cancer, kidney cancer, liver cancer, lung cancer, melanoma, ovarian cancer, pancreatic cancer, prostate cancer, or stomach cancer.

8: The method of claim 1, wherein the sample is a biofluid sample selected from serum or plasma.

9: The method of claim 1, wherein the immunoassay is a radioimmunoassay, fluorescence immunoassay, or an enzyme-linked immunosorbent assay.
